(19) United States
(12) Patent Application Publication
(10) Pub. No.: US 2021/0287762 A1
(43) Pub. Date: Sep. 16, 2021

(54) MOLECULAR SIMILARITY SEARCH

(71) Applicant: GSI Technology Inc., Sunnyvale, CA (US)

(72) Inventor: Elona EREZ, Tel Aviv (IL)

(21) Appl. No.: 17/200,836

(22) Filed: Mar. 14, 2021

Related U.S. Application Data
(60) Provisional application No. 62/989,937, filed on Mar. 16, 2020.
(60) Provisional application No. 63/150,597, filed on Feb. 18, 2021.

(51) Int. Cl. G16C 20/40; G16C 20/70; G06F 16/903; G06N 3/08

(52) U.S. Cl. CPC G16C 20/40; G06N 3/08; G06F 16/90335; G16C 20/70

(57) ABSTRACT

A system for finding similar molecules to a query molecule includes a GCN, a PFS vector extractor, a compensated vector comparator (CVC) and a candidate vector selector. The GCN has been trained to output a molecular property vector from an input query or input candidate molecular vectors, respectively, The GCN transforms query atomic feature set (AFS) vectors and candidate AFS vectors into query property feature set (PFS) embedding vectors and candidate PFS embedding vectors. The PFS vector extractor extracts query PFS embedding vectors and candidate PFS embedding vectors from hidden layers of the trained GCN. The compensated vector comparator (CVC) calculates a compensated similarity metric (CSM) for at least one pair of query PFS embedding vector and one candidate PFS embedding vector. The candidate vector selector selects only such candidate molecular vectors.

### CROSS REFERENCE TO RELATED APPLICATIONS

This application claims priority from U.S. provisional patent applications 62/989,937 filed Mar. 16, 2020 and 63/150,597 filed on Feb. 18, 2021, which are incorporated herein by reference.

### FIELD OF THE INVENTION

The present invention relates to similarity search generally and to molecular similarity search in particular.

### BACKGROUND OF THE INVENTION

One of the mainstays of the drug industry is small-molecule-drugs. Pharmaceutical researchers search for the molecule that will, for example, inhibit an enzyme or activate a receptor in the way they desire. Using artificial intelligence (AI) for molecular property prediction is known.

Drug makers use molecular similarity search to try to predict properties such as solubility—how well a molecule can dissolve into the blood or enter the membrane of a cell; toxicity—the degree to which a molecule can damage an organism; and, blood brain barrier (BBB)—does the molecule enter the brain or not. After first screening of a molecule for structure, researchers employ deep learning techniques to find molecules with similar desired properties as known molecules.

Researchers utilize Neural Networks which are mathematical models, in this case convolutional neural networks (CNN) or graphical convolution networks (GCN) to recognize the properties of molecules. These may be implemented on software platforms such as Rdkit, Deepchem and others.

Reference is now made to FIGS. 1A and 1B, which illustrate a GCN 1 comprising multiple neural layers; an input layer 2, a plurality of hidden layers 3, and an output layer 4. Each layer comprises a plurality of nodes 6, and the nodes in each layer may be connected by a plurality of connections 7. Each node may be fully connected to each node in the previous and subsequent layers, but is not required to be as such.

An input vector Vᵢ, representing the structure and atomic features of a molecule, as described in detail hereinbelow, enters GCN 1 at input layer 2, and traverse hidden layers 3 and an output vector Vₒ exits GCN 1 at output layer 4.

There are two main modes of operating an GCN: training mode and operational mode (which includes testing, verification and regular use of GCN 1). During training, input vectors Vᵢ, with an output value of Vₒ which is known, are put through GCN 1. The nodes 6, weights W, connections 7 and other features of GCN 1 explained further hereinbelow, are adjusted, for example by cross entropy loss, so when V₁ traverses GCN 1, GCN 1 transforms Vᵢ to equal the known value of Vₒ at output layer 4. Training a GCN to perform accurate transformations is a complex task, as is known in the art.

Once a GCN is trained, another set of input vectors is used to test and verify if the GCN transformation is reliable and accurate. Another set of test input vectors, again with known output values is passed through GCN 1 and actual Vₒ results are compared against known Vₒ values. If the results are acceptable, the GCN is considered trained. Once trained, the GCN may be used to predict the output of unknown query vectors.

Researchers strive to create the perfect transformation model, within a GCN, that will generate a desired output for a given input. For example, structural and atomic properties, called features, of a molecule, may be input to a GCN, and the toxicological properties of such a molecule may be predicted at the output. As known by those in the art, during the training phase of a GCN, various deep learning techniques are used to refine the GCN. These techniques include, but are not limited to neighbor feature aggregation layers, normalization layers, pooling layers, non-linear transformation layers, readout layers, and others. Current GCN techniques are described in the website publication, Deep Learning, at http://www.deeplearningbook.org; in the article “SimGNN: A Neural Network Approach to Fast Graph Similarity Computation” published by ACM 2019; and, Semi-Supervised Classification with Graph Convolutional Networks published by ICLR 2017.

Using the toxicology example mentioned hereinabove, the U.S. Environmental Protection Agency, the U.S. National Toxicology Program, the U.S. National Center for Advancing Translational Sciences, and the U.S. Food and Drug Administration formed the Tox21 Consortium that created the Tox21 molecular property dataset. The Tox21 dataset comprises: a database of over 12,000 molecules used to train, validate and test GCNs. Training molecules have a known set of 12 toxicological properties that are used by GCN 1 during training, to self-adjust nodes 6, connections 7, weights W and other GCN features mentioned hereinabove, to train the GCN to output the correct Tox21 12-bit property set for a given input molecule.

The Tox21 dataset has sets of input vectors, with known output vectors that can be used to train GCN 1. Other sets of vectors are included in the dataset for testing and verification. In total there are about 12,000 vectors available. The training molecule set is chosen to reflect the range of input types used with GCN 1. Likewise, validation vectors are a set of molecules that will test the full breadth of the performance of the GCN, but are not used during training. Finally, when GCN 1 has been tested and validated, unknown molecular vectors are input to GCN 1 and their Tox21 properties predicted at output 4.

Reference is now made to FIGS. 2A and 2B which illustrate the input and output vectors of GCN 1. Each input vector Vᵢ, comprises atomic feature sets (AFS) 10 for all s atoms in the molecule, and a spatial data file (SDF) 11. Each AFS 10 describes one atom in the input molecule and comprises 128 features. SDF 11 defines the structure and adjacency of the atoms within the molecule, and is used by GCN 1 to factor in the effects of neighboring atoms.

The output vector Vₒ is a 12-bit binary vector representing the Tox21 molecular properties 13 of the molecule. These 12 properties are divided into a 7-bit ‘nuclear receptor panel’ of seven toxicological properties: (1) estrogen receptor alpha, LBD (ER, LBD); (2) estrogen receptor alpha, full (ER, full); (3) aromatase; (4) aryl hydrocarbon receptor (AhR); (5) androgen receptor, full (AR, full); (6) androgen receptor, LBD (AR, LBD); (7) peroxisome proliferator-activated receptor gamma (PPAR-gamma), and a 5-bit ‘stress response panel’ of 5 toxicological properties: (8) nuclear factor (erythroid-derived 2)-like 2/antioxidant responsive element (Nrf2/ARE); (9) heat shock factor response element (HSE); (10) ATADS; (11) mitochondrial membrane potential (MMP); (12) p53.

### SUMMARY OF THE PRESENT INVENTION

There is provided in accordance, with a preferred embodiment of the present invention, a method for finding similar molecules to a query molecule. The method includes transforming query atomic feature set (AFS) vectors and candidate AFS vectors into query property feature set (PFS) embedding vectors and candidate PFS embedding vectors, utilizing a GCN that has been trained to output a molecular property vector from an input query or input candidate molecular vectors, respectively. The method also includes extracting query and candidate PFS embedding vectors from hidden layers of the trained GCN, calculating a compensated similarity metric (CSM) for at least one pair of the query PFS embedding vector and one candidate PFS embedding vector, and selecting only such candidate molecular vectors which have a value of the CSM above a pre-defined threshold value.

Moreover, in accordance with a preferred embodiment of the present invention, compensating attempts to compensate for inaccuracies caused by a varying position of atomic feature sets at the input layer of the trained GCN.

Further, in accordance with a preferred embodiment of the present invention, calculating includes, for each candidate PFS embedding vector, summing all possible combinations of dot products between property feature sets in the query PFS embedding vector and property feature sets in the candidate PFS embedding vector, and normalizing the dot product sum, by dividing the dot product sum by the number of the property feature sets in the candidate PFS embedding vector.

Still further, in accordance with a preferred embodiment of the present invention, the trained GCN includes an input layer, four hidden layers and an output layer.

Additionally, in accordance with a preferred embodiment of the present invention, each PFS embedding vector includes a plurality of property feature sets.

Moreover, in accordance with a preferred embodiment of the present invention, the trained GCN is trained to one of the following properties: solubility, blood brain barrier or toxicity.

Further, in accordance with a preferred embodiment of the present invention, extracting query and candidate PFS embedding vectors is performed at the output of the fourth hidden layer.

Still further, in accordance with a preferred embodiment of the present invention, the candidate AFS vectors are vectors used to train the GCN.

Additionally, in accordance with a preferred embodiment of the present invention, adjusting the predefined threshold value changes the number of candidate molecular vectors deemed similar to the query molecular vector.

There is also provided, in accordance with a preferred embodiment of the present invention, a system for finding similar molecules to a query molecule. The system includes a GCN, a PFS vector extractor, a compensated vector comparator (CVC), and a candidate vector selector. The GCN has been trained to output a molecular property vector from an input query or input candidate molecular vectors, respectively, The GCN transforms query atomic feature set (AFS) vectors and candidate AFS vectors into query property feature set (PFS) embedding vectors and candidate PFS embedding vectors. The PFS vector extractor extracts query PFS embedding vectors and candidate PFS embedding vectors from hidden layers of the trained GCN. The compensated vector comparator (CVC) calculates a compensated similarity metric (CSM) for a pair of one query PFS embedding vector and one candidate PFS embedding vector. The candidate vector selector selects only such candidate molecular vectors which have a value of the CSM above a pre-defined threshold value.

Additionally, in accordance with a preferred embodiment of the present invention, the compensated vector comparator (CVC) attempts to compensate for inaccuracies caused by a varying position of atomic feature sets at the input layer of the trained GCN.

Further, in accordance with a preferred embodiment of the present invention, the CVC includes a dot product summer and a DPS normalizer. The dot product summer sums all possible combinations of dot products between property feature sets in the query PFS embedding vector and property feature sets in the candidate PFS embedding vector, for each candidate PFS embedding vector. The DPS normalizer normalizes the DPS, by dividing the DPS by the number of property feature sets in the candidate PFS embedding vector, for each candidate PFS embedding vector.

Still further, in accordance with a preferred embodiment of the present invention, the candidate vector selector changes the value of the predefined threshold value in order to change the number of candidate molecular vectors deemed similar to the query molecular vector.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are illustrations of a GCN comprising multiple neural layers;

FIGS. 2A and 2B are illustrations of the input and output vectors of a GCN;

FIG. 3 is an illustration of a toxicology molecular similarity search system;

FIG. 4 is an illustration of the layers in an embodiment of a trained GCN;

FIG. 5A is an illustration of a TFS embedding vector;

FIG. 5B is an illustration of a compensated vector comparator (CVC);

FIG. 6A is an illustration of an exemplary query TFS embedding vector;

FIG. 6B is an illustration of an example of the sum of TFS dot products;

FIG. 7 is an illustration of a general molecular similarity search system

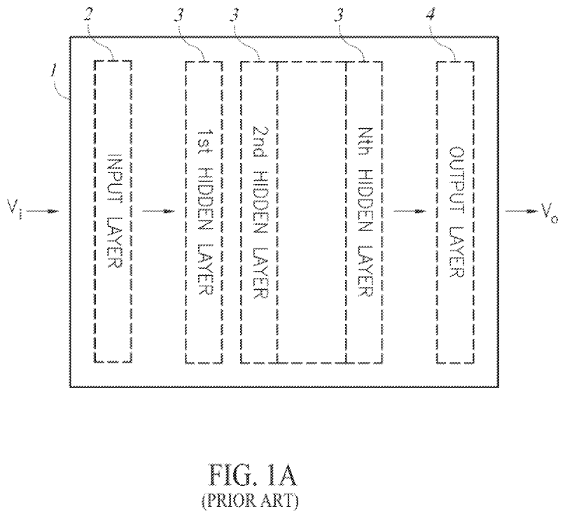

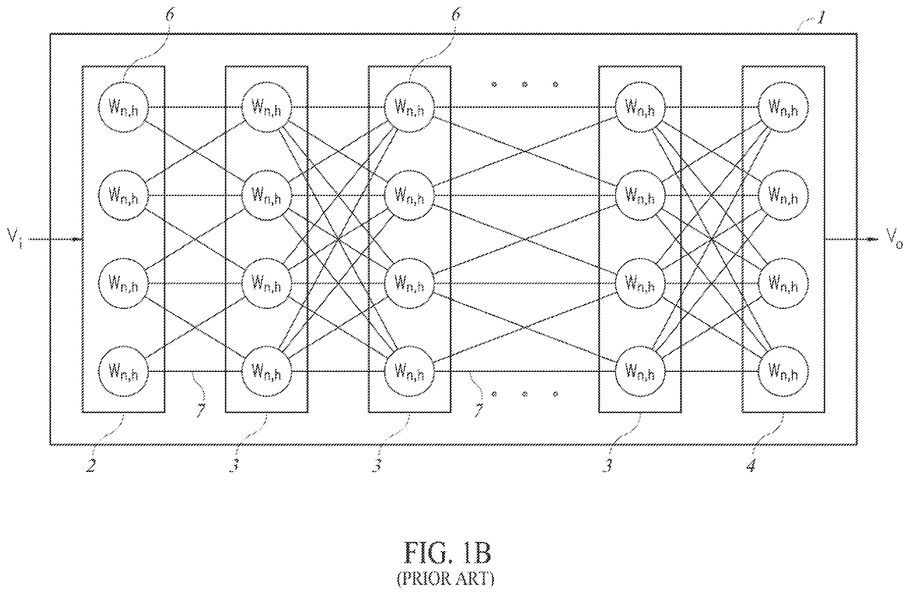

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that in a toxicologically trained Graphical Convolution Networks (GCNs), as the input vector, comprising a plurality of atomic feature sets (AFS), traverses from the input layer and through a plurality of hidden layers, its AFS data are transformed to toxicological feature set (TFS) data, before being further transformed into the toxicology property vector at the output layer.

Applicant has realized that this is not only true for toxicology, but also for other molecular properties such as blood brain barrier (BBB), solubility and other properties. In such GCNs that are trained according to a particular molecular property, as input vectors traverse the GCN, AFS data is transformed to property feature sets (PFS) before being further transformed into the appropriate property vector at the output layer. The present application uses toxicology as an example.

Applicant has also realized that, rather than use the toxicology output vector from such toxicological GCNs, TFS embedding vectors may be extracted from within the hidden layers of the GCN and used outside of the GCN to mathematically compare their toxicological properties with other extracted TFS embedding vectors.

Applicant has realized that the order that atoms are presented to the input layer of a GCN may affect the output accuracy. For example, a water molecule AFS vector having two hydrogen atoms and one oxygen atom may be presented to the GCN input layer as H—H—O, H—O—H or O—H—H.

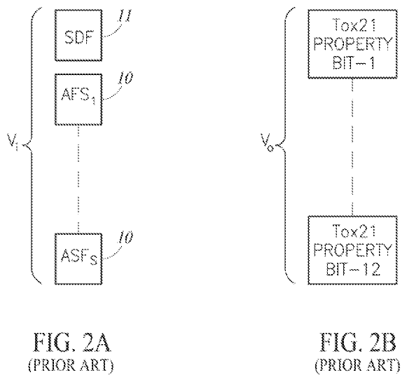

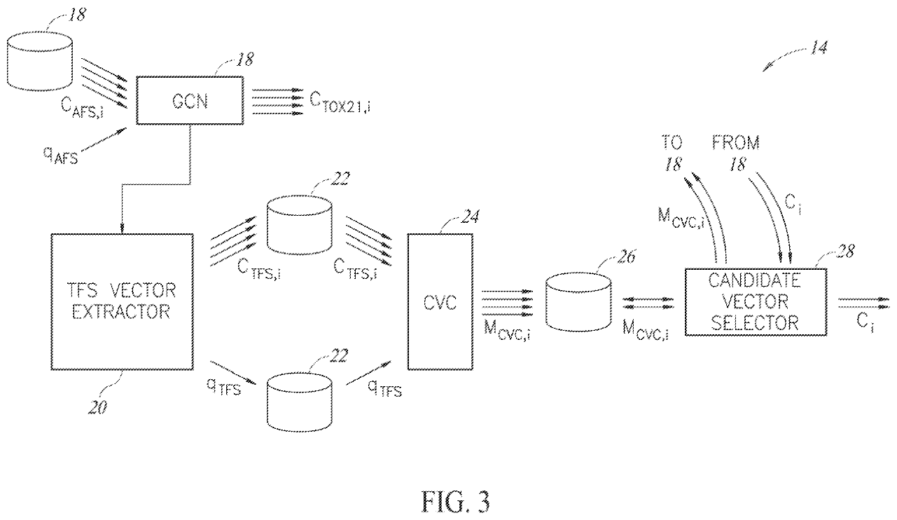

Reference is now made to FIG. 3 which illustrates a molecular similarity search system 14 comprising a GCN 16 that has been trained using the Tox21 dataset, a toxicology molecule candidate database 18 containing, for example, Tox21 molecular vectors c_{AFS,i} (as described in FIG. 2A), a toxicity feature set (TFS) vector extractor 20 to extract a query TFS embedding vector q_{TFS} and multiple candidate TFS embedding vectors c_{TFS,i} from within GCN 16, a TFS embedding vector database 22 to store TFS embedding vectors q_{TFS} and c_{TFS,i}, a compensated vector comparator (CVC) 24 to calculate a compensated similarity metric (CSM) M_{cvc,i}, between TFS embedding vectors q_{TFS} and c_{TFS,i}, that minimizes the effect of the order of atomic data in q_{AFS} and c_{AFS}, a CSM database 26 to store CSM M_{cvc,i}, and a candidate vector selector 28 to select those candidate vectors c_{AFS,i} deemed similar to query vector q_{AFS}.

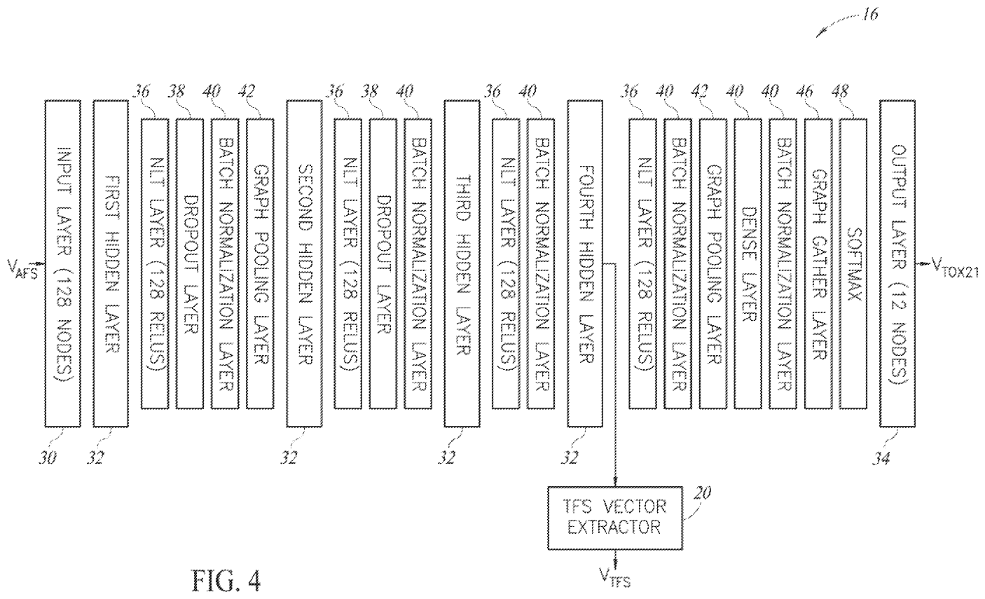

Any GCN may be utilized, for example. Reference is now made to FIG. 4 which illustrates the layers in an embodiment of trained GCN 16 shown in FIG. 3. GCN 16 may be configured with an input layer 30 containing 128 nodes, four hidden layers 32 also containing 128 nodes, and an output layer 34 containing 12 nodes. GCN 16 of FIG. 4 utilizes 4 hidden layers to calculate in the effects of neighboring atoms as defined in the SDF file, mentioned herein above. At input layer 30, the calculation takes into account the atomic feature sets of only the molecular atoms alone. For example, if H—O—H is presented at input layer 30 only the effects of the feature set of H are calculated at the first node, and only the effects of O are calculated at the second node, and only the effects of the second H are calculated at the third node.

At first hidden layer 32, the effects of feature sets of first-degree neighboring atoms are also calculated. At the first node, H—O is included, at the second node H—O—H, and at the third node O—H. At the third hidden layer 32, the secondary neighbors are included, which are H—O—H on the first node and H—O—H on the third node and at the fourth hidden layer 32, the tertiary neighbor are included. There are no tertiary neighbors in the H₂O example, but in the Tox21 dataset, each molecule has about 20 atoms, and the neighboring atoms may have a greater effect on the calculation.

As mentioned hereinabove, there are many deep learning techniques that are applied within a GCN to improve the performance and accuracy of the GCN. In the preferred embodiment of the present invention, on the output of the first hidden layer 32 there is: a non-linear translation (NLT) layer 36 containing 128 Relus; a dropout layer 38 set to 0.1; a batch normalization layer 40; and, a graph pooling layer 42 set to max pool over the feature vectors for an atom and its neighbors in bond-graph. On the output of the second hidden layer 32 there is: a non-linear translation (NLT) layer 36 containing 128 Relus; a dropout layer 38 set to 0.1; a batch normalization layer 40. On the output of the third hidden layer 32 there is: a non-linear translation (NLT) layer 36 containing 128 Relus; and, a batch normalization layer 40; and on the output of the fourth hidden layer 32 there is: a non-linear translation (NLT) layer 36 containing 128 Relus, batch normalization 40; a graph pooling layer 42; a dense layer 44; another batch normalization layer 40; a graph gather layer 46; and a Softmax layer 48.

It will be appreciated that the specific techniques employed, the number of layers and the number of nodes in GCN 16 may vary and are presented here as examples of configuring a neural network.

Applicant has realized that vectors in the Tox21 dataset may be used not only for training GCNs, but to produce candidate TFS embedding vectors c_{TFS,i} with which to compare with query TFS embedding vector q_{TFS}.

Returning to FIG. 3, molecular similarity search system 14 takes candidate vectors c_{AFS} from toxicology molecule candidate database 18 containing for example, about 12000 Tox21 molecular sample vectors, and passes them through toxicologically trained GCN 16. TFS vector extractor 20 extracts candidate TFS embedding vectors c_{TFS,i} from the output of fourth hidden layer 32 (as shown in FIG. 4) before any output conditioning layers mentioned hereinabove, and then stores them in TFS embedding vector database 22. Query vector q_{AFS} is also input to GCN 16 and TFS vector extractor 20 may extract query TFS embedding vector q_{TFS} and may store it in TFS embedding vector database 22.

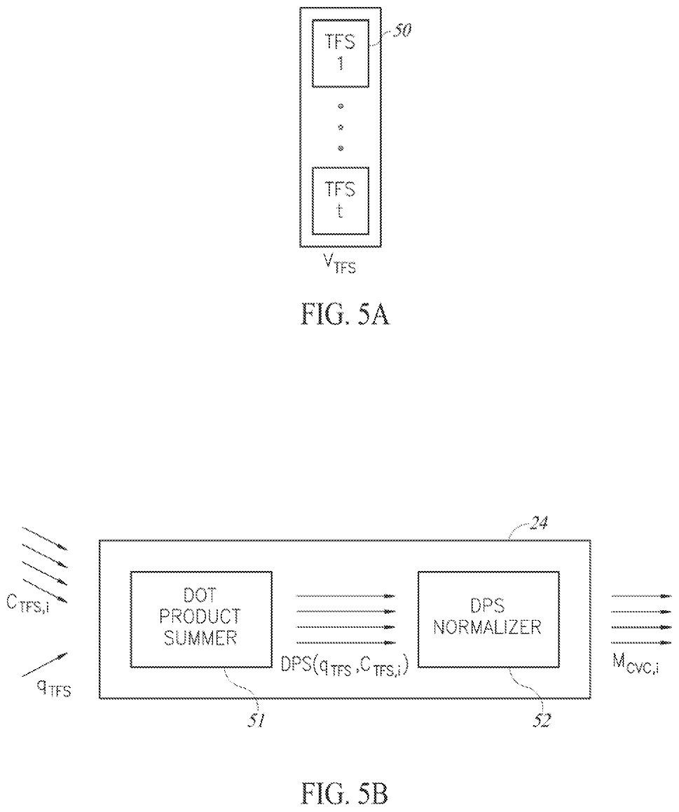

Reference is briefly made to FIG. 5A which illustrates a TFS embedding vector V_{TFS} which could be a candidate vector c_{TFS,i} or a query vector q_{TFS}. TFS embedding vectors comprise a plurality of TFSs 50 one for each of t atoms in the molecular vector. Such TFS embedding vectors may be stored in TFS embedding vector database 22.

Applicant has realized that the arrangement of atomic feature sets in input vectors V_{AFS} may also affect the arrangement of toxicity feature sets in TFS embedding vectors V_{TFS}. Applicant has also realized that calculations performed on TFS embedding vectors V_{TFS} need to compensate for the effects of such TFS arrangements in TFS embedding vectors V_{TFS}. Applicant has realized that in the toxicology example, by using the normalized sum of TFS dot products between embedding vector pairs as a metric, such positioning effects are minimized and more accurate similarity metric for vector pairs can be calculated.

Reference is now made to FIG. 5B which illustrates CVC 24 comprising a dot product summer 51 and a dot product sum normalizer 52. dot product summer 51 may take query TFS embedding vectors q_{TFS} and candidate TFS embedding vectors c_{TFS,i} from TFS embedding vector database 22 and calculate a dot product sum of the vectors.

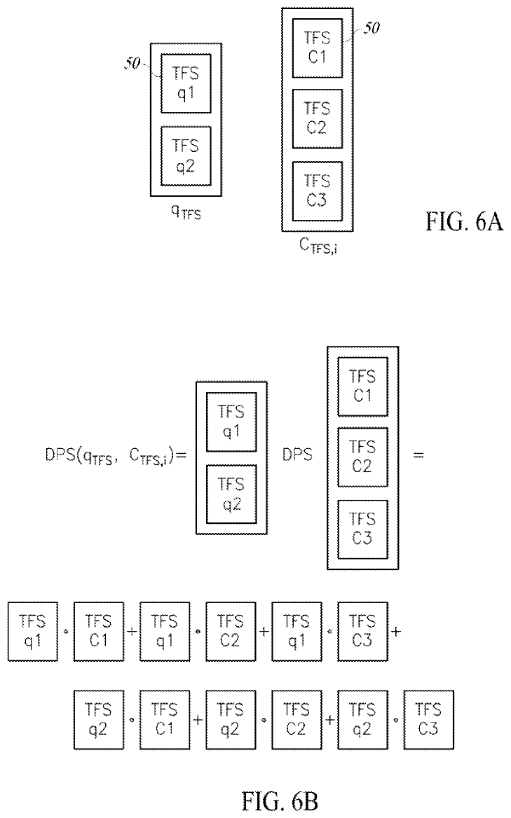

Reference is now made to FIG. 6A which illustrates exemplary query TFS embedding vector q_{TFS} and candidate TFS embedding vector c_{TFS,i}. Query TFS embedding vector q_{TFS} comprises two toxicity feature sets 50—TFS_{q1} and TFS_{q2}, and candidate TFS embedding vector c_{TFS,i} comprises three toxicity feature sets 50—TFS_{c1}, TFS_{c2} and TFS_{c3}. Reference is now made to FIG. 6B which illustrates an example of the sum of TFS dot products between embedding vectors q_{TFS} and c_{TFS,i}. Dot product summer 51 calculates the sum of all dot products DPS(q_{TFS}, c_{TFS,i}) for all combinations of query TFS embedding vector q_{TFS} toxicity feature sets 50 and candidate TFS embedding vector c_{TFS,i} toxicity feature sets 50 as shown in FIG. 6B and in equation (1):

DPS(q_{TFS}, c_{TFS,i})=[TFS_{q1}·TFS_{c1}]+[TFS_{q1}·TFS_{c2}]+[TFS_{q1}·TFS_{c3}]+[TFS_{q2}·TFS_{c1}]+[TFS_{q2}·TFS_{c2}]+[TFS_{q2}·TFS_{c3}] equation (1)

dot product sum normalizer 52 then completes the CSM calculation by normalizing DPS(q_{TFS}, c_{TFS,i}), by dividing it by the number of atoms tin the candidate vector c_{TFS,i} (which in the example is 3), as shown in equation (2):

M_{CVC,i}=Normalized DPS(q_{TFS}, c_{TFS,i})=[DPS(q_{TFS}, c_{TFS,i})]/t equation (2)

CVC 24 then stores each M_{CVC,i} for each TFS query-candidate pair q_{TFS}−c_{TFS,i} in CSM database 26. M_{CVC,i} is then used by candidate vector selector 28 as a score against which it then selects only those candidate vectors C_{AFS,i} with a score over a candidate score threshold. Those candidates with a score over such a threshold are deemed similar to query vector q_{AFS}.

It should be noted that the embodiments described hereinabove may be implemented on any suitable computing device. All databases may be implemented as individual databases or sections of a single database. Extracted TFS embedding vectors may be used for any calculation, not only similarity metrics as shown hereinabove. TFS embedding vectors may be extracted from GCNs trained with any training vector set, not only toxicity vectors as shown hereinabove.

Applicant has also realized that by enabling candidate vector selector 28 to adjust the threshold score by which candidates are deemed similar, users have the flexibility to adjust the size of the candidate pool, without having to retrain the neural network.

Applicant has also realized that calculations can be implemented as simple Boolean functions, and performed in parallel on all candidate vectors simultaneously on associative memory arrays such as Gemini Associative Processing Unit, commercially available from GSI Technologies Inc. of the USA.

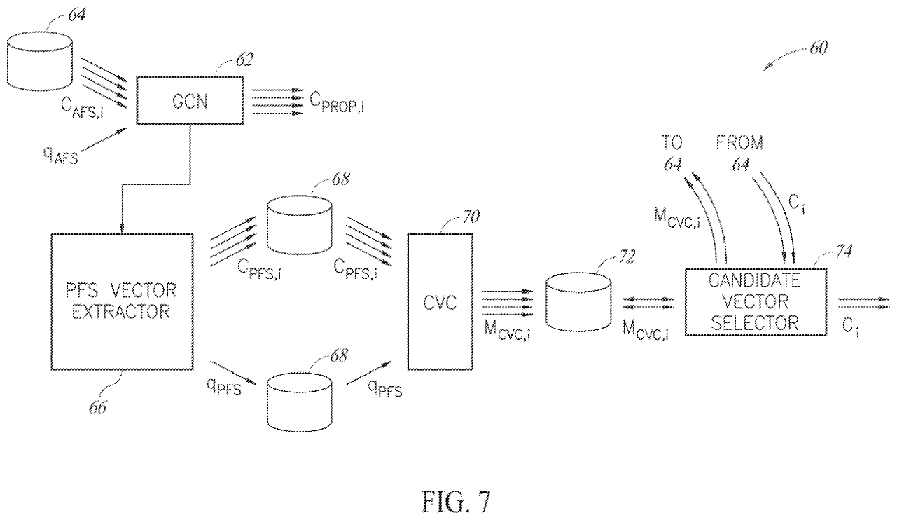

As mentioned hereinabove, such a GCN could be trained using any molecular property, such as solubility, BBB or other property. Reference is now made to FIG. 7 which illustrates a general molecular similarity search system 60 comprising a GCN 62 that has been trained using any known molecular property, a molecule candidate database 64 containing molecular vectors c_{AFS,i} (as described in FIG. 2A), a property feature set (PFS) vector extractor 66 to extract a query PFS embedding vector q_{PFS} and multiple candidate PFS embedding vectors c_{PFS,i} from within GCN 62, a PFS embedding vector database 68 to store PFS embedding vectors q_{PFS} and c_{PFS,i}, a compensated vector comparator (CVC) 70 to calculate a compensated similarity metric (CSM) M_{cvc,i}, between PFS embedding vectors q_{PFS} and c_{PFS,i}, that attempts to minimizes the effect of the order of atomic data in q_{AFS} and c_{AFS}, a CSM database 72 to store CSM M_{cvc,i}, and a candidate vector selector 74 to select those candidate vectors c_{AFS,i} deemed similar to query vector q_{AFS}.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a general purpose computer of any type, such as a client/server system, mobile computing devices, smart appliances, cloud computing units or similar electronic computing devices that manipulate and/or transform data within the computing system's registers and/or memories into other data within the computing system's memories, registers or other such information storage, transmission or display devices.

Embodiments of the present invention may include apparatus for performing the operations herein. This apparatus may be specially constructed for the desired purposes, or it may comprise a computing device or system typically having at least one processor and at least one memory, selectively activated or reconfigured by a computer program stored in the computer. The resultant apparatus when instructed by software may turn the general-purpose computer into inventive elements as discussed herein. The instructions may define the inventive device in operation with the computer platform for which it is desired. Such a computer program may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus. The computer readable storage medium may also be implemented in cloud storage.

Some general-purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for finding similar molecules to a query molecule, the method comprising:
transforming query atomic feature set (AFS) vectors and candidate AFS vectors into query property feature set (PFS) embedding vectors and candidate PFS embedding vectors, utilizing a GCN that has been trained to output a molecular property vector from an input query or input candidate molecular vectors, respectively;
extracting query and candidate PFS embedding vectors from hidden layers of said trained GCN;
calculating a compensated similarity metric (CSM) for at least one pair of said query PFS embedding vector and one said candidate PFS embedding vector; and
selecting only such said candidate molecular vectors which have a value of said CSM above a pre-defined threshold value.

2. The method according to claim 1 wherein said compensating attempts to compensate for inaccuracies caused by a varying position of said atomic feature sets at an input layer of said trained GCN.

3. The method according to claim 1 wherein said calculating comprises:
for each candidate PFS embedding vector:
summing all possible combinations of dot products between property feature sets in said query PFS embedding vector and property feature sets in said candidate PFS embedding vector; and
normalizing said dot product sum, by dividing said dot product sum by the number of said property feature sets in said candidate PFS embedding vector.

4. The method according to claim 1 wherein said trained GCN comprises an input layer, four hidden layers and an output layer.

5. The method according to claim 1 wherein each said PFS embedding vector comprises a plurality of property feature sets.

6. The method according to claim 1 wherein said trained GCN is trained to one of the following properties: solubility, blood brain barrier and toxicity.

7. The method according to claim 4 wherein said extracting query and candidate PFS embedding vectors is performed at the output of the fourth said hidden layer.

8. The method according to claim 1 wherein said candidate AFS vectors are vectors used to train said GCN.

9. The method according to claim 1 wherein adjusting said predefined threshold value changes the number of said candidate molecular vectors deemed similar to said query molecular vector.

10. A system for finding similar molecules to a query molecule, the system comprising:
a GCN that has been trained to output a molecular property vector from an input query or input candidate molecular vectors, respectively, to transform query atomic feature set (AFS) vectors and candidate AFS vectors into query property feature set (PFS) embedding vectors and candidate PFS embedding vectors;
a PFS vector extractor to extract query PFS embedding vectors and candidate PFS embedding vectors from hidden layers of said trained GCN;
a compensated vector comparator (CVC) to calculate a compensated similarity metric (CSM) for at least one pair of said query PFS embedding vector and one said candidate PFS embedding vector; and
a candidate vector selector to select only such said candidate molecular vectors which have a value of said CSM above a pre-defined threshold value.

11. The system according to claim 10 wherein said compensated vector comparator (CVC) attempts to compensate for inaccuracies caused by a varying position of said atomic feature sets at an input layer of said trained GCN.

12. The system according to claim 11 wherein said CVC comprises:
a dot product summer to sum all possible combinations of dot products between property feature sets in said query PFS embedding vector and property feature sets in said candidate PFS embedding vector, for each candidate PFS embedding vector; and
a DPS normalizer to normalize said DPS, by dividing said DPS by the number of said property feature sets in said candidate PFS embedding vector, for each candidate PFS embedding vector.

13. The system according to claim 10 wherein said trained GCN comprises an input layer, four hidden layers and an output layer.

14. The system according to claim 10 wherein each said PFS embedding vector comprises a plurality of property feature sets.

15. The system according to claim 10 wherein said trained GCN is trained to one of the following properties: solubility, blood brain barrier and toxicity.

16. The system according to claim 13 wherein said PFS vector extractor extracts query and candidate PFS embedding vectors from the output of the fourth said hidden layer.

17. The system according to claim 10 wherein said candidate AFS vectors are vectors used to train said GCN.

18. The system according to claim 10 wherein said candidate vector selector to change the value of said predefined threshold value in order to change the number of said candidate molecular vectors deemed similar to said query molecular vector.
